(19) United States
(12) Patent Application Publication
(10) Pub. No.: US 2024/0024443 A1
(43) Pub. Date: Jan. 25, 2024

(54) AUGMENTATION OF SURVIVIN MODIFIED MRNA VACCINE EFFICACY USING DENDRITIC CELLS

(71) Applicant: Regen Biopharma, Inc., La Mesa, CA (US)

(72) Inventors: Thomas ICHIM, San Diego, CA (US); David KOOS, La Mesa, CA (US)

(73) Assignee: Regen Biopharma, Inc., La Mesa, CA (US)

(21) Appl. No.: 18/358,432

(22) Filed: Jul. 25, 2023

Related U.S. Application Data
(60) Provisional application No. 63/391,889, filed on Jul. 25, 2022.

(51) Int. Cl. A61K 39/00

(52) U.S. Cl. CPC A61K 39/00115; A61K 2039/53; A61K 2039/54; A61K 2039/55522; A61K 2039/55527; A61K 2039/55516; A61K 2039/55533

(57) ABSTRACT

Disclosed are methods, and compositions useful for stimulation of immunity towards cancer by utilizing dendritic cells and products therefrom that have been stimulated and/or incorporated mRNA encoding survivin and/or related gene sequences, defined herein as “survivin modified mRNA”. In one embodiment dendritic cells generated from allogeneic “off the shelf” sources are transfected with survivin modified mRNA and the cells are administered in an immunogenic manner. In some embodiments transfected dendritic cells are induced to undergo immunogenic cell death ex vivo subsequently to which they are used as immunogenic stimuli. In other embodiments preparation of the injection site is performed prior to administration of dendritic cells that have been transfected with survivin modified mRNA by pre-administration of agents increasing the number of local dendritic cells in the skin. In embodiments in vivo, transfection of survivin modified mRNA is performed by administration of dendritic cell accumulating agents followed by in vivo transfection.

### CROSS-REFERENCE TO RELATED APPLICATIONS

This application claims priority to U.S. Provisional Application No. 63/391,889, titled “Augmentation of Survivin Modified mRNA Vaccine Efficacy Using Dendritic Cells” and filed Jul. 25, 2022, which is hereby incorporated by reference in its entirety.

### FIELD OF THE INVENTION

The invention relates to the field of cancer treatment and more specifically utilizing dendritic cells and products therefrom that have been stimulated and/or incorporated mRNA encoding survivin and/or related gene sequences.

### BACKGROUND OF THE INVENTION

Currently modified mRNA vaccines have demonstrated significant efficacy in terms of induction of immunity to a variety of antigens. Most notably, modified mRNA vaccination has been cited as the major breakthrough allowing for rapid immunization to SARS-CoV-2 during the COVID-19 pandemic.

Despite previous attempts at using mRNA vaccination strategies in cancer, to date, no FDA cleared cancer mRNA vaccine is available. The current invention seeks to overcome this deficiency in the art.

### SUMMARY

Preferred methods include embodiments of preventing or treating cancer through administration of one or more mRNA molecules encoding the whole or a portion of the survivin gene, wherein said mRNA are survivin modified mRNA.

Preferred embodiments include methods wherein the modified mRNA contains at least one chemically modified nucleoside selected from the group consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinom-ethyluridine, 1-taurinomethyl-pseudouridine, 5-taurinom-ethyl-2-thio-uridine, 1-taurinomethyl-4-thio-uridine, 5-methyl-uridine, 1-methylpseudouridine, 4-thio-1-methyl-pseudouridine, 2-thio-1-methyl-pseudouridine, 1-methyl-1-deaza-pseudouridine, 2-thio-1-methyl-1-deaza-pseudouri-dine, dihydrouridine, dihydropseudouridine, 2-thio-dihydrouridine, 2-thio-dihydropseudouridine, 2-methoxyuridine, 2-methoxy-4-thio-uridine, 4-methoxypseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thiozebularine, 2-thio-zebularine, 2-methoxy-cytidine, 2-methoxy-5-methyl-cytidine, 4-methoxy-pseudoisocytidine 4-methoxy-1-methy 1-pseudoisocytidine, 2-aminopurine, 2,6-diaminopurine, 7-deaza-adenine, 7-deaza-8-azaadenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyl-adenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deazaguanosine, 6-thio-7-deaza-8-aza-guanosine, 7-methylguanosine, 6-thio-7-methyl-guanosine, 7-methylinosine, 6-methoxy-guanosine, 1-methylguanosine, N2-methyl-guanosine, N2,N2-dimethylguanosine, 8-oxo-guanosine, 7-methyl-8-oxo-guanosine, l-methyl-6-thio-guanosine, N2-methyl-6-thio-guanosine, and N2,N2-dimethyl-6-thio-guanosine.

Preferred methods include embodiments wherein said administration is performed by subcutaneous injection of said mRNA molecules.

Preferred methods include embodiments wherein said administration of said mRNA molecules is performed into the lymph nodes.

Preferred methods include embodiments wherein agents are administered prior to administration of mRNA in order to enhance immunogenicity of molecules transcribed from said mRNA.

Preferred embodiments include methods wherein said agent capable of enhancing immunogenicity of said molecules transcribed from said mRNA is G-CSF.

Preferred methods include embodiments wherein said agent capable of enhancing immunogenicity of said molecules transcribed from said mRNA is GM-CSF.

Preferred methods include embodiments wherein said agent capable of enhancing immunogenicity of said molecules transcribed from said mRNA is M-CSF.

Preferred methods include embodiments wherein said agent capable of enhancing immunogenicity of said molecules transcribed from said mRNA is interleukin-3.

Preferred methods include embodiments wherein said agent capable of enhancing immunogenicity of said molecules transcribed from said mRNA is M-CSF and interferon gamma.

Preferred methods include embodiments wherein said agent capable of enhancing immunogenicity of said molecules transcribed from said mRNA is GM-CSF and interferon gamma.

Preferred methods include embodiments wherein said agent capable of enhancing immunogenicity of said molecules transcribed from said mRNA is G-CSF and interferon gamma.

Preferred methods include embodiments wherein said agent capable of enhancing immunogenicity of said molecules transcribed from said mRNA is G-CSF and interferon gamma and antibody to interleukin-10.

Preferred methods include embodiments wherein said agent capable of enhancing immunogenicity of said molecules transcribed from said mRNA is GM-CSF and interferon gamma and antibody to interleukin-10.

Preferred methods include embodiments wherein said agent capable of enhancing immunogenicity of said molecules transcribed from said mRNA is M-CSF and interferon gamma and antibody to interleukin-10.

Preferred methods include embodiments wherein said agent capable of enhancing immunogenicity of said molecules transcribed from said mRNA is M-CSF and antibody to interleukin-10.

Preferred methods include embodiments wherein said agent capable of enhancing immunogenicity of said molecules transcribed from said mRNA is G-CSF and antibody to interleukin-10.

Preferred methods include embodiments wherein said agent capable of enhancing immunogenicity of said molecules transcribed from said mRNA is GM-CSF and antibody to interleukin-10.

Preferred methods include embodiments wherein said agent capable of enhancing immunogenicity of said molecules transcribed from said mRNA is interleukin-4.

Preferred methods include embodiments wherein said agent capable of enhancing immunogenicity of said molecules transcribed from said mRNA is interleukin-2.

Preferred methods include embodiments wherein said agent capable of enhancing immunogenicity of said molecules transcribed from said mRNA is interleukin-6.

Preferred methods include embodiments wherein said agent capable of enhancing immunogenicity of said molecules transcribed from said mRNA is interleukin-7.

Preferred methods include embodiments wherein said agent capable of enhancing immunogenicity of said molecules transcribed from said mRNA is interleukin-12.

Preferred methods include embodiments wherein said agent capable of enhancing immunogenicity of said molecules transcribed from said mRNA is TNF-alpha.

Preferred methods include embodiments wherein said agent capable of enhancing immunogenicity of said molecules transcribed from said mRNA is lymphotoxin.

Preferred methods include embodiments wherein said agent capable of enhancing immunogenicity of said molecules transcribed from said mRNA is interleukin-15.

Preferred methods include embodiments wherein said agent capable of enhancing immunogenicity of said molecules transcribed from said mRNA is interleukin-16.

Preferred methods include embodiments wherein said agent capable of enhancing immunogenicity of said molecules transcribed from said mRNA is interleukin-18.

Preferred methods include embodiments wherein said agent capable of enhancing immunogenicity of said molecules transcribed from said mRNA is interleukin-21.

Preferred methods include embodiments wherein said agent capable of enhancing immunogenicity of said molecules transcribed from said mRNA is interleukin-23.

Preferred methods include embodiments wherein said agent capable of enhancing immunogenicity of said molecules transcribed from said mRNA is interleukin-27.

Preferred methods include embodiments wherein said agent capable of enhancing immunogenicity of said molecules transcribed from said mRNA is interleukin-33.

Preferred methods include embodiments wherein said agent capable of enhancing immunogenicity of said molecules transcribed from said mRNA is interleukin-15 and interleukin-18.

Preferred methods include embodiments wherein said agent capable of enhancing immunogenicity of said molecules transcribed from said mRNA is a small molecule ERK inhibitor.

Preferred methods include embodiments wherein said ERK inhibitor is PD0325901.

Preferred methods include embodiments wherein said ERK inhibitor is RDEA119.

Preferred methods include embodiments wherein said ER3K inhibitor is Olomoucine.

Preferred methods include embodiments wherein said ERK inhibitor is Aminopurvalanol A.

Preferred methods include embodiments wherein said ERK inhibitor is AS703026.

Preferred methods include embodiments wherein said ERK inhibitor is AZD8330.

Preferred methods include embodiments wherein said ERK inhibitor is BIX02188.

Preferred methods include embodiments wherein said ERK inhibitor is BIX02189.

Preferred methods include embodiments wherein said ERK inhibitor is CI-1040.

Preferred methods include embodiments wherein said ERK inhibitor is Cobimetirlib.

Preferred methods include embodiments wherein said ERK inhibitor is GDC-0623.

Preferred methods include embodiments wherein said ERK inhibitor is MEk162.

Preferred methods include embodiments wherein survivin gene mRNA is administered into dendritic cells which are subsequently administered to the patient.

Preferred methods include embodiments wherein said dendritic cells are generated from monocytes.

Preferred methods include embodiments wherein said monocytes plastic adherent.

Preferred methods include embodiments wherein said monocytes express CD14.

Preferred methods include embodiments wherein said monocytes express CD16.

Preferred methods include embodiments wherein said monocytes express TLR4.

Preferred methods include embodiments wherein said monocytes express TNF alpha upon stimulation.

Preferred methods include embodiments wherein said monocytes express CD90.

Preferred methods include embodiments wherein said monocytes express c-kit.

Preferred methods include embodiments wherein said monocytes express c-met.

Preferred methods include embodiments wherein said monocytes express CD25.

Preferred methods include embodiments wherein said monocytes express PDGF-receptor.

Preferred methods include embodiments wherein said monocytes express CD16.

Preferred methods include embodiments wherein said monocytes express BDNF-receptor.

Preferred methods include embodiments wherein said monocytes are treated with IL-4 and GM-CSF ex vivo to generate immature dendritic cells.

Preferred methods include embodiments wherein said immature dendritic cells express IL-10.

Preferred methods include embodiments wherein said immature dendritic cells express CD11c.

Preferred methods include embodiments wherein said immature dendritic cells express lower levels of CD40 as compared to mature dendritic cells.

Preferred methods include embodiments wherein said immature dendritic cells express lower levels of CD80 as compared to mature dendritic cells.

Preferred methods include embodiments wherein said immature dendritic cells express lower levels of CD86 as compared to mature dendritic cells.

Preferred methods include embodiments wherein said immature dendritic cells express lower levels of IL-12 as compared to mature dendritic cells.

Preferred methods include embodiments wherein said immature dendritic cells express lower levels of IL-21 as compared to mature dendritic cells.

Preferred methods include embodiments wherein said immature dendritic cells express lower levels of IL-18 as compared to mature dendritic cells.

Preferred methods include embodiments wherein said immature dendritic cells express lower levels of IL-33 as compared to mature dendritic cells.

Preferred methods include embodiments wherein said immature dendritic cells express lower levels of IL-15 as compared to mature dendritic cells.

Preferred methods include embodiments wherein said immature dendritic cells express higher levels of IL-35 as compared to mature dendritic cells.

Preferred methods include embodiments wherein said immature dendritic cells express higher levels of TGF-beta as compared to mature dendritic cells.

Preferred methods include embodiments wherein said immature dendritic cells express higher levels of HLA-G as compared to mature dendritic cells.

Preferred methods include embodiments wherein said immature dendritic cells express lower levels of AIM2 as compared to mature dendritic cells.

Preferred methods include embodiments wherein said immature dendritic cells express higher levels of ILT-3 as compared to mature dendritic cells.

Preferred methods include embodiments wherein said immature dendritic cells express higher levels of ILT-4 as compared to mature dendritic cells.

Preferred methods include embodiments wherein said immature dendritic cells express higher levels of LIF as compared to mature dendritic cells.

Preferred methods include embodiments wherein said immature dendritic cells express higher levels of inhibitor of kappa B as compared to mature dendritic cells.

Preferred methods include embodiments wherein said immature dendritic cells express higher levels of soluble TNF-alpha receptor as compared to mature dendritic cells.

Preferred methods include embodiments wherein said immature dendritic cells express higher levels of interleukin-1 receptor antagonist as compared to mature dendritic cells.

Preferred methods include embodiments wherein said dendritic cells are induced to mature after administration in vivo.

Preferred methods include embodiments wherein said maturation is induced by administration of Poly (IC).

Preferred methods include embodiments wherein said maturation is induced by administration of imiquimod.

Preferred methods include embodiments wherein said maturation is induced by administration of HMGB-1.

Preferred methods include embodiments wherein said maturation is induced by administration of CpG motifs.

Preferred methods include embodiments wherein said maturation is induced by administration of xenogeneic cell membranes.

Preferred methods include embodiments wherein said maturation is induced by administration of bacterial cell wall extract.

Preferred methods include embodiments wherein said maturation is induced by administration of beta glucan.

Preferred methods include embodiments wherein said maturation is induced by administration of OK231.

Preferred methods include embodiments wherein said maturation is induced by administration of GM-CSF.

Preferred methods include embodiments wherein said maturation is induced by administration of neutrophil extracellular traps.

Preferred methods include embodiments wherein said maturation is induced by administration of free histones.

Preferred methods include embodiments wherein said maturation is induced by administration of yeast cell wall extract.

Preferred methods include embodiments wherein said maturation is induced by administration of KLH.

Preferred methods include embodiments wherein said maturation is induced by administration of zymosan.

Preferred methods include embodiments wherein said maturation is induced by administration of interferon gamma.

Preferred methods include embodiments wherein said maturation is induced by administration of antibodies to IL-10 or its receptor.

Preferred methods include embodiments wherein said maturation is induced by administration of TNF-alpha.

Preferred methods include embodiments wherein said maturation is induced by administration of IL-33.

Preferred methods include embodiments wherein said maturation is induced by administration of beta defensin.

Preferred methods include embodiments wherein said maturation is induced by administration of complement C3.

Preferred methods include embodiments wherein said maturation is induced by administration of complement C5.

Preferred methods include embodiments wherein said maturation is induced by administration of necrotic cells.

Preferred embodiments include methods of treating cancer comprising: a) transfecting a pluripotent stem cell with expression vectors inducibling upregulated survivin expression; b) differentiating said pluripotent stem cells into dendritic cells; c) administering said dendritic cells into a cancer patient; and d) inducing expression of survivin in said dendritic cell by administration of an inducer molecule.

Preferred methods include embodiments wherein said survivin modified mRNA consists of a survivin encoding mRNA and a portion of mRNA encoding sections of the HMGB-1 gene.

Preferred methods include embodiments wherein said fusion mRNA construct encodes in part the amino acid sequence SAFFLFCSE (SEQ ID NO.: 1)

Preferred methods include embodiments wherein said fusion mRNA construct encodes in part the amino acid sequence DPNAPKRPPSAFFL. (SEQ ID NO.: 2)

Preferred methods include embodiments wherein said fusion mRNA construct encodes in part the amino acid sequence RPPSAFFLL. (SEQ ID NO.: 3)

### DETAILED DESCRIPTION OF THE INVENTION

The invention teaches the utilization of mRNA vaccination to survivin in the context of dendritic cell upregulation of immunity. General embodiments include transfection of dendritic cells in vitro with survivin before administration, in vivo transfection of dendritic cells, and means of engineering dendritic cells to induce and/or boost immunity to the survivin mRNA vaccine.

Unless defined differently, all technical and scientific terms used herein have the same meanings as commonly understood by one of skill in the art to which the disclosed invention belongs. In particular, the following terms and phrases have the following meaning.

:Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, /pindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “polypeptide” is used interchangeably with “peptide”, “altered peptide ligand”, and “flourocarbonated peptides.”

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha..beta.T cell expressing TCR .alpha. and .beta. chains, and a .gamma..delta.T cell expressing TCR .gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used.

The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

The invention provides the utilization of dendritic cells as a means to stimulate the enhanced immunogenicity of mRNA vaccination to survivin. To assist one in skill of the art to practice the invention, we provide several examples of traditional DC vaccines. The practice of the invention teaches the use of mRNA to transfect cells of the DC origin for use as immunogens. Numerous animal models have demonstrated that in the context of neoplasia DCs can bind to and engulf tumour antigens that are released from tumor cells, either alive or dying, and cross-present these antigens to T cells in tumour-draining lymph nodes. This results in the generation of tumour-specific immune responses that have been demonstrated to inhibit tumor growth or in some cases induced transferrable immunological memory. Mechanistically, DCs recognize tumors using the same molecular means that they would use to recognize apoptotic cells, or cells that are stressed. One set of signals are molecules released from apoptotic cells, which are highly released by tumors, these include the nucleotides UTP and ATP, fractalkine, lipid lysophosphatidylcholine, and sphingosine 1-phosphate [1]. Signals from stressed cells, such as tumor cells include externalization of phosphatidylserine onto the outside of the cell membrane, calreticulin, avß5 integrin, CD36 and lactadherin. There is some evidence that dendritic cells actively promote tumor immunity in that patients with dendritic cell infiltration of tumors generally have a better prognosis [2-5]. While DC themselves are part of the initial immune response to cancer, numerous mechanisms are used by tumors to suppress the ability of DC to stimulate an immune response. One particular mechanism is the depletion of tryptophan in the tumor microenvironment by production of indolamine 2,3 deoxygenase (IDO) [6], which will be discussed in detail in Section 5.6. Tryptophan depletion results in T cell apoptosis, while catabolites of tryptophan depletion are known to lead to suppression of T cell activation. In order to overcome issues associated with local tumor suppression of DC, numerous studies have utilized ex vivo generated DC that have been pulsed with tumor antigen as a means of stimulating anticancer immunity.

The most advanced DC based therapy is the product Provenge (sipuleucel-T), which is approved by the FDA for treatment of androgen resistant prostate cancer. Provenge is a cellular product derived from autologous peripheral blood mononuclear cell (PBMC) derived dendritic cells that have been grown using a chimeric protein comprised of GM-CSF and the prostate specific antigen, prostatic acid phosphatase [7, 8]. In the pivotal trial, this DC based therapeutic resulted in extension of survival by 4.1 months [8]. Prior to approval of Provenge, numerous clinical trials using DC were performed in prostate cancer, which will be discussed below. Tjoa et al reported on 33 participants of a phase I trial in patients with advanced prostate cancer that received autologous DC pulsed HLA-A0201-specific prostate-specific membrane antigen (PSMA) peptides (PSM-P1 or -P2) that were entered into a second trial (Phase II) which involved six infusions of DC pulsed with PSM-P1 and -P2 peptides. The patients were followed up for up to 770 days from the start of the original phase I study. 9 partial responders were identified in the phase II study based on National Prostate Cancer Project (NPCP) criteria, plus 50% reduction of prostate-specific antigen. Four of the partial responders were also responders in the phase I study, with an average response duration of 225 days. Their combined average total response period was over 370 days. Five other responders in the secondary immunizations at the Phase II were nonresponders in the phase I study. Their average partial response period was 196 days. These data support the safety of follow-up infusion of DC that have been pulsed with tumor antigen derived peptide [9]. The same group published a subsequent paper on an additional 33 patients that had not received prior DC immunization in the Phase I. All subjects received six infusions of DC pulsed with PSM-P1 and -P2 at six week intervals without any treatment associated adverse events. Six partial and two complete responders were identified in the phase II study based on NPCP criteria, plus 50% reduction of prostate-specific antigen (PSA), or resolution in previously measurable lesions on ProstaScint scan [10]. The same group analyzed immune response in patients who had clinical remission or relapsed. A strong correlation was found between delayed type hypersensitivity response to the PSM-P1 and PSM-P2 and clinical response [11].

The approach that was to evolve into Provenge was described in a paper that reported outcome of 12 androgen resistant prostate cancer patients treated with DC that were pulsed with a GM-CSF-PAP fusion protein. Two intravenous infusions of the generated cells were performed one month apart. The infusions were followed by three s.c. monthly doses of the fusion protein without cells. Treatment was well tolerated and circulating prostate-specific antigen levels dropped in three patients. Immune response to the fusion protein was observed, as well as to PAP [12]. A subsequent study utilized the Provenge approach as used today, that is, without the subcutaneous boosting with protein alone. In this study, DC precursors were harvested by leukapheresis in weeks 0, 4, 8, and 24, loaded ex vivo with antigen for 2 days, and then infused intravenously over 30 minutes. Phase I patients received increasing doses of Provenge, and phase II patients received all the Provenge that could be prepared from a leukapheresis product. Patients tolerated treatment well. Fever, the most common adverse event, occurred after 15 infusions (14.7%). All patients developed immune responses to the recombinant fusion protein used to prepare Provenge, and 38% developed immune responses to PAP. Three patients had a more than 50% decline in prostate-specific antigen (PSA) level, and another three patients had 25% to 49% decreases in PSA. The time to disease progression correlated with development of an immune response to PAP and with the dose of dendritic cells received [13]. An additional study utilized the same approach to treat 21 patients with histologically documented androgen-independent prostate carcinoma that could be evaluated by radionuclide bone scan or computed tomography scan. Provenge was prepared from a leukapheresis product; it contained autologous CD54-positive recombinant GM-CSF-PAP loaded DC with admixtures of monocytes, macrophages, B and T cells. Provenge was infused intravenously twice, 2 weeks apart. Two weeks after the second infusion, patients received three subcutaneous injections of 1.0 mg of the recombinant protein 1 month apart. Nineteen patients could be evaluated for response to treatment. The median time to progression was 118 days. Treatment was tolerated reasonably well; most adverse effects were secondary to Provenge and were NCI Common Toxicity Criteria Grade 1-2. Four of the 21 patients reported Grade 3-4 adverse events. Two patients exhibited a transient 25-50% decrease in prostate-specific antigen (PSA). For a third patient, PSA dropped from 221 ng/ml at baseline to undetectable levels by week 24 and has remained so for more than 4 years. In addition, this patient's metastatic retroperitoneal and pelvic adenopathy has resolved. PBMC collected from patients for at least 16 weeks proliferated upon in vitro stimulation by the recombinant GM-CSF-PAP. For the patient with responsive disease, PBMC could be stimulated for 96 weeks [14]. Another study assessed the effects of Provenge on androgen independent prostate cancer with biochemical progression. This type of cancer is earlier in the oncogenesis process as compared to androgen resistant cancer. Specifically, patients with nonmetastatic recurrent disease as manifested by increasing PSA levels (0.4-6.0 ng/mL) and who had undergone previous definitive surgical or radiation therapy were enrolled. Therapy consisted of Provenge infusions on weeks 0, 2, and 4 (ie, 3 infusions). Prostate-specific antigen was measured at baseline and monthly until disease progression, defined as a doubling of the baseline or nadir PSA value (whichever was lower) to > or =4 ng/mL or development of distant metastases. Thirteen of 18 patients demonstrated an increase in PSA doubling time (PSADT), with a median increase of 62% (4.9 months before treatment vs. 7.9 months after treatment; P=0.09; signed-rank test). These data suggested that Provenge has therapeutic activity both on androgen dependent, which is more early stage, and androgen dependent, which is more late stage, prostate cancer [15]. The Phase III trial for Provenge consisted of 512 randomly assigned prostate cancer patients in a 2:1 ratio to receive either Provenge (341 patients) or placebo (171 patients) administered intravenously every 2 weeks, for a total of three infusions. The primary end point was overall survival, analyzed by means of a stratified Cox regression model adjusted for baseline levels of serum prostate-specific antigen (PSA) and lactate dehydrogenase. In the Provenge group, there was a relative reduction of 22% in the risk of death as compared with the placebo group (P=0.03). This reduction represented a 4.1-month improvement in median survival (25.8 months in the Provenge group vs. 21.7 months in the placebo group). The 36-month survival probability was 31.7% in the Provenge group versus 23.0% in the placebo group. Immune responses to the immunizing antigen were observed in patients who received Provenge but not controls [16].

In addition to Provenge, which as mentioned above, received FDA marketing approval, several other types of antigens have been utilized in DC therapy of prostate cancer. For example, while PSA is a known biochemical marker of prostate cancer progression, the PSA protein or peptides from this protein have been identified to possess immunogenic properties. One study examined the possibility of utilizing PSA protein pulsed DC for treatment of prostate cancer. Twenty-four patients with histologically proven prostate carcinoma and an isolated postoperative rise of serum PSA (>1 ng/ml to 10 ng/ml) after radical prostatectomy were included. The patients received nine administrations of PSA-loaded DCs by combined intravenous, subcutaneous, and intradermal routes over 21 weeks. Circulating prostate cancer cells detected in six patients at baseline were undetectable at 6 months and remained undetectable at 12 months. Eleven patients had a postbaseline transient PSA decrease on one to three occasions, predominantly occurring at month 1 (7 patients) or month 3 (2 patients). Maximum PSA decrease ranged from 6% to 39%. PSA decrease on at least one occasion was more frequent in patients with low Gleason score (p=0.016) at prostatectomy and with positive skin tests at study baseline (p=0.04). PSA-specific T cells were detected ex vivo by ELISpot for IFN-gamma in 7 patients before vaccination and in 11 patients after vaccination. Of the latter 11 patients, 5 had detectable T cells both before and during the vaccination period, 4 only during the vaccination period, while 2 patients could for technical reasons not be assessed prevaccination. No induction of anti-PSA IgG or IgM antibodies was detected. There were no serious adverse events or otherwise severe toxicities observed during the trial [17]. Proteins may possess both immune stimulatory and immune inhibitory epitopes. Thus some studies sought to utilize specific peptides which are known to be immune stimulatory. Accordingly, a clinical trial was conducted in 28 patients with locally advanced or metastatic prostate cancer to determine whether an HLA-A2 binding epitope of prostate-specific antigen, PSA146-154 (PSA-peptide), can induce specific T cell immunity. Patients were vaccinated either by intradermal injection of PSA-peptide and GM-CSF or by intravenous administration of autologous dendritic cells pulsed with PSA-peptide at weeks 1, 4 and 10. DTH skin testing was performed at weeks 4, 14, 26 and 52. Fifty percent of the patients developed positive DTH responses to PSA-peptide. Cytokine analysis of PSA-peptide stimulated T cells exhibited specific IFN-gamma and TNF-alpha response in six of seven patients. Specific IL-4 response was observed in five patients, while IL-10 response was detected in one patient. Purified CD4-CD8+ T cells isolated from four patients demonstrated specific cytolytic activity per chromium release assay. This trial demonstrated that, immunization with PSA-peptide induced specific T cell immunity in one-half of the patients with locally advanced and hormone-sensitive, metastatic prostate cancer. DTH-derived T cells exhibited PSA-peptide-specific cytolytic activity and predominantly expressed a type-1 cytokine profile [18]. A subsequent study sought to boost effects of PSA peptide pulsed DC through administration of interferon gamma in the treatment of 12 hormone resistant prostate cancer patients. All patients were vaccinated four times with intracutaneously injected PSA-peptide loaded DCs after subcutaneous administration of IFN-gamma 2 hr before DC administration (50 microg/m(2) body surface). The vaccination was well tolerated without any vaccination-associated adverse events. One partial and one mixed responder were identified, four patients showed stable diseases. Two patients had a decrease and four a slow-down velocity slope in the PSA serum level. All responders showed a positive DTH-response, but only two a slight increase in PSA-peptide specific T-lymphocytes [19]. Given that tumors may suppress expression of certain peptides, or alternatively may mutate the peptide, a more global approach towards stimulation of anticancer immunity has been the utilization of multiple peptides to overcome this hurdles. A clinical study in 8 androgen resistant prostate cancer patients utilized a cocktail consisting of HLA-A*0201-restricted peptides derived from five different prostate cancer-associated antigens [prostate-specific antigen (PSA), prostate-specific membrane antigen (PSMA), survivin, prostein, transient receptor potential p8 (trp-p8)]. Patients were treated with 4 vaccinations of pulsed DC once every two weeks. Apart from local skin reactions no side effects were noted. One patient displayed a partial response (PR; PSA decrease >50%) and three other patients showed stable PSA values or decelerated PSA increases. In ELISPOT analyses, three of four PSA responders also showed antigen-specific CD8+ T-cell activation against prostein, survivin, and PSMA [20]. Cocktail approaches have been used by other investigators using different peptides, for example, a study by Waeckerle-Men et al. utilized autologous DC of HLA-A*0201(+) patients with hormone-refractory prostate cancer that were loaded with antigenic peptides derived from prostate stem cell antigen (PSCA(14-22)), prostatic acid phosphatase (PAP(299-307)), prostate-specific membrane antigen (PSMA(4-12)), and prostate-specific antigen (PSA(154-163)). DC were intradermally applied six times at biweekly intervals followed—in the case of an enhanced immune response—by monthly booster injections. Of the three patients that were reported, the vaccination elicited significant cytotoxic T cell responses against all prostate-specific antigens tested. In addition, memory T cell responses against the control peptides derived from influenza matrix protein and tetanus toxoid were efficiently boosted. Clinically, the long-term DC vaccination was associated with an increase in PSA doubling time [21]. In addition to prostate cancer, in which FDA approval has been granted for the Provenge drug, numerous trials have been conducted in a wide variety of cancers. All the trials demonstrated safety, without serious adverse effects of DC administration, as well as some degree of therapeutic efficacy. Trials have been conducted in melanoma [22-73], soft tissue sarcoma [74], thyroid [75-77], glioma [78-99], multiple myeloma, [100-108], lymphoma [109-111], leukemia [112-119], as well as liver [120-125], lung [126-139], ovarian [140-143], and pancreatic cancer [144-146].

In some embodiments of the invention, the invention provides means of utilizing the potent antigen presenting activities of the dendritic cell together with tumor specific marker survivin. The leveraging of survivin targeting is also useful because it is a marker found on tumor endothelial cells. In some embodiments the dendritic cells are generated in vitro from iPSC. In some embodiments, survivin mRNA is used to destroy tumor angiogenic processes. The angiogenic activity in cancer is a very dynamic and fluid process. Although VEGF is critical in formation of new blood vessels, there are multiple other cytokines that possess redundant roles. Many times resistance to VEGF signaling is a result of tumor endothelial cells becoming more reliant on other cytokines and factors such as survivn that take over the function of VEGF pathway [52, 53]. Additionally, mutations in VEGF-R2 have been identified in tumor endothelium, which has been associated with non-responsiveness [54]. In situations like this, targeting of survivin is useful. Dose escalation of VEGF inhibiting antibodies is limited by different toxicities such as cardiac toxicities [55-61]. An interesting observation is that although hematopoietic stem cells are known to utilize VEGF-R2 for self-renewal, cytopenias are generally not observed in patients receiving VEGF pathway blockers [62, 63]. Kinase inhibitors also suffer from mutations of active sites, as well as off target toxicities. For example, a study in colon patients receiving sunitinib demonstrated mutations in all major kinases associated with endothelial proliferation [64].

Thus limitations of efficacy of anti-angiogenesis approaches that are currently in the clinic appear to be associated with: a) targeting of only one pathway allows the tumor endothelium to start utilizing other pathways; and b) small molecule inhibitors are slow in onset of action, which allows for time to pass and mutations to accumulate.

Immunological targeting of survivin mediated angiogenesis may be a more promising approach due to: a) ability of immune system to “mutate” with cancer endothelium, thus overcoming ability of molecular evasion; b) more rapid onset of immune attack, including direct killing of endothelium may not allow enough time for tumor endothelium to mutate and/or acquire resistant properties.

Classical studies have shown that tumor infiltrating lymphocytes correlate with positive prognosis in various tumors [65-77]. The invention teaches means of increasing lymphocytic recognition of cancer endothelial cells. Unfortunately, there are several important factors that prevent efficacy of infiltrating lymphocytes. Firstly, tumor masses originate from tumor stem cells, which possess distinctly different antigenic composition [78-82]. Accordingly, infiltration of lymphocytes, while useful for targeting tumor stem cell progeny, may not actually reach, or recognize tumor stem cells. This is also relevant in light of studies showing tumor stem cells possess various immune evasive molecules such as DAF, IL-10 and HLA-G. Secondly, tumors are known to possess high interstitial pressure, which physically limits ability of lymphocytes to enter the tumor mass, which often possesses necrotic tissue. Thirdly, tumor acidosis, hypoxia, and high adenosine concentrations have been demonstrated to selectively inhibit cytotoxic cells and promote T regulatory cells. In one embodiment depletion of T regulatory cells is disclosed together with survivin mRNA vaccination. Despite theoretical obstacles to efficacy of immunotherapy of tumors, numerous studies have shown that in some patients, cancer vaccines, ranging from whole cell lysates of the 1970s to defined peptide vaccines, to nucleic acid vaccines, all induce in select patients some level of tumor regression. While in double blind trials these approaches often fail, due to heterogeneity of patients and tumors, in some patients documented durable responses are observed. It is not the scope of this paper to speculate on the heterogeneity, however, factors, which were not appreciated in previous studies, including polymorphisms in immune associated genes, expression of different mutations, diet, and gut microbiota content may all contribute observations of tumor eradication in some patients whereas in other patients no effect or even acceleration of tumor. Regardless of cause, it is documented that some patients highly respond to active vaccination against tumor antigens.

We propose that significantly higher killing of tumor cells may be achieved by directing antigen-specific immune responses toward the tumor endothelium by targeting of survivin. In contrast, to tumor stem cells, which are the desired target of an effective vaccination program, tumor endothelial cells are directly in contact with the circulatory system, thus permitting uninhibited access to immune cells and antibodies. The invention discloses the previously unknown observation that cancer stem cells overexpress survivin and that targeting survivin is a novel means of depletion cancer stem cells. Additionally, tumor endothelium is known to possess an increased level of prothrombotic molecules such as tissue factor [83-87]. The invention teaches that targeting survivin decreases tumor thrombogenic activities. Thus hypothetically, stimulation of thrombosis may be induced at a reduced threshold by immune cells/molecules targeting tumor angiogenesis as compared to existing vasculature in the body, which possesses numerous antithrombotic activities.

Early studies demonstrated that xenogeneic immunization with angiogenic proteins resulted in tumor regression. In some embodiments of the invention iPSC derived survivin overexpressing cells are utilized in a xenogeneic manner. In other embodiments xenogeneic survivin is utilized to induce immunity. Breaking of self-tolerance using xenogeneic proteins is commonly used to elicit autoimmunity in models such as collagen induced arthritis and experimental autoimmune encephalitis (EAE). Accordingly, previous studies have shown that while inhibition of tolerance to self-proteins associated with oncogenic angiogenesis results in inhibition of tumor growth, alterations to physiological processes such as wound healing or menstruation where not observed. This perhaps indicates the ability of the immune system to differentiate between pathological conditions of angiogenesis versus physiological. One potential analogy are clinical studies using antigen specific T cells for autoimmunity as a vaccine. In these studies, despite T cells being administered in an immunogenic manner, only antigen specific, idiotypic T cells are immunologically attacked and not all T cells.

In some embodiments survivin immunization is utilized together with stimulation of immunity to molecules associated with tumor blood vessels have been utilized therapeutically in animal models for vaccination purposes including survivin [103-105], endosialin [106], and xenogeneic FGF2R [107], VEGF [108], VEGF-R2 [109], MMP-2 [110], and endoglin [111, 112].

### REFERENCES

1. Ravichandran, K. S., Beginnings of a good apoptotic meal: the find-me and eat-me signaling pathways. Immunity, 2011. 35(4): p. 445-55.
2. Hu, M., et al., Decreased intratumoral Foxp3 Tregs and increased dendritic cell density by neoadjuvant chemotherapy associated with favorable prognosis in advanced gastric cancer. Int J Clin Exp Pathol, 2014. 7(8): p. 4685-94.
3. Ayari, C., et al., High level of mature tumor-infiltrating dendritic cells predicts progression to muscle invasion in bladder cancer. Hum Pathol, 2013. 44(8): p. 1630-7.
4. Liska, V., et al., Infiltration of colorectal carcinoma by S100+ dendritic cells and CD57+ lymphocytes as independent prognostic factors after radical surgical treatment. Anticancer Res, 2012. 32(5): p. 2129-32.
5. Ayari, C., et al., Bladder tumor infiltrating mature dendritic cells and macrophages as predictors of response to bacillus Calmette-Guerin immunotherapy. Eur Urol, 2009. 55(6): p. 1386-95.
6. Johnson, T. S. and D. H. Munn, Host indoleamine 2,3-dioxygenase: contribution to systemic acquired tumor tolerance. Immunol Invest, 2012. 41(6-7): p. 765-97.
7. Sternberg, C. N., et al., Progress in the treatment of advanced prostate cancer. Am Soc Clin Oncol Educ Book, 2014: p. 117-31.
8. Gomella, L. G., F. Gelpi-Hammerschmidt, and C. Kundavram, Practical guide to immunotherapy in castration resistant prostate cancer: the use of sipuleucel-T immunotherapy. Can J Urol, 2014. 21(2 Supp 1): p. 48-56.
9. Tjoa, B. A., et al., Evaluation of phase I/II clinical trials in prostate cancer with dendritic cells and PSMA peptides. Prostate, 1998. 36(1): p. 39-44.
10. Murphy, G. P., et al., Infusion of dendritic cells pulsed with HLA-A2-specific prostate-specific membrane antigen peptides: a phase II prostate cancer vaccine trial involving patients with hormone-refractory metastatic disease. Prostate, 1999. 38(1): p. 73-8.
11. Lodge, P. A., et al., Dendritic cell-based immunotherapy of prostate cancer: immune monitoring of a phase II clinical trial. Cancer Res, 2000. 60(4): p. 829-33.
12. Burch, P. A., et al., Priming tissue-specific cellular immunity in a phase I trial of autologous dendritic cells for prostate cancer. Clin Cancer Res, 2000. 6(6): p. 2175-82.
13. Small, E. J., et al., Immunotherapy of hormone-refractory prostate cancer with antigen-loaded dendritic cells. J Clin Oncol, 2000. 18(23): p. 3894-903.
14. Burch, P. A., et al., Immunotherapy (APC8015, Provenge) targeting prostatic acid phosphatase can induce durable remission of metastatic androgen-independent prostate cancer: a Phase 2 trial. Prostate, 2004. 60(3): p. 197-204.
15. Beinart, G., et al., Antigen-presenting cells 8015 (Provenge) in patients with androgen-dependent, biochemically relapsed prostate cancer. Clin Prostate Cancer, 2005. 4(1): p. 55-60.
16. Kantoff, P. W., et al., Sipuleucel-T immunotherapy for castration-resistant prostate cancer. N Engl J Med, 2010. 363(5): p. 411-22.
17. Barrou, B., et al., Vaccination of prostatectomized prostate cancer patients in biochemical relapse, with autologous dendritic cells pulsed with recombinant human PSA. Cancer Immunol Immunother, 2004. 53(5): p. 453-60.
18. Perambakam, S., et al., Induction of specific T cell immunity in patients with prostate cancer by vaccination with PSA146-154 peptide. Cancer Immunol Immunother, 2006. 55(9): p. 1033-42.
19. Hildenbrand, B., et al., Immunotherapy of patients with hormone-refractory prostate carcinoma pre-treated with interferon-gamma and vaccinated with autologous PSA-peptide loaded dendritic cells—a pilot study. Prostate, 2007. 67(5): p. 500-8.
20. Fuessel, S., et al., Vaccination of hormone-refractory prostate cancer patients with peptide cocktail-loaded dendritic cells: results of a phase I clinical trial. Prostate, 2006. 66(8): p. 811-21.
21. Waeckerle-Men, Y., et al., Dendritic cell-based multi-epitope immunotherapy of hormone-refractory prostate carcinoma. Cancer Immunol Immunother, 2006. 55(12): p. 1524-33.
22. Nestle, F. O., et al., Vaccination of melanoma patients with peptide-or tumor lysate-pulsed dendritic cells. Nat Med, 1998. 4(3): p. 328-32.
23. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
24. Wang, F., et al., Phase I trial of a MART-1 peptide vaccine with incomplete Freund's adjuvant for resected high-risk melanoma. Clin Cancer Res, 1999. 5(10): p. 2756-65.
25. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
26. Thomas, R., et al., Immature human monocyte-derived dendritic cells migrate rapidly to draining lymph nodes after intradermal injection for melanoma immunotherapy. Melanoma Res, 1999. 9(5): p. 474-81.
27. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
28. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.
29. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
30. Lau, R., et al., Phase I trial of intravenous peptide-pulsed dendritic cells in patients with metastatic melanoma. J Immunother, 2001. 24(1): p. 66-78.
31. Banchereau, J., et al., Immune and clinical responses in patients with metastatic melanoma to CD34(+) progenitor-derived dendritic cell vaccine. Cancer Res, 2001. 61(17): p. 6451-8.
32. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
33. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
34. Bedrosian, I., et al., Intranodal administration of peptide-pulsed mature dendritic cell vaccines results in superior CD8+T-cell function in melanoma patients. J Clin Oncol, 2003. 21(20): p. 3826-35.
35. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
36. Hersey, P., et al., Phase I/II study of treatment with dendritic cell vaccines in patients with disseminated melanoma. Cancer Immunol Immunother, 2004. 53(2): p. 125-34.
37. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
38. Palucka, A. K., et al., Spontaneous proliferation and type 2 cytokine secretion by CD4+ T cells in patients with metastatic melanoma vaccinated with antigen-pulsed dendritic cells. J Clin Immunol, 2005. 25(3): p. 288-95.
39. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
40. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
41. Salcedo, M., et al., Vaccination of melanoma patients using dendritic cells loaded with an allogeneic tumor cell lysate. Cancer Immunol Immunother, 2006. 55(7): p. 819-29.
42. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
43. Escobar, A., et al., Dendritic cell immunizations alone or combined with low doses of interleukin-2 induce specific immune responses in melanoma patients. Clin Exp Immunol, 2005. 142(3): p. 555-68.
44. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
45. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
46. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
47. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
48. Palucka, A. K., et al., Dendritic cells loaded with killed allogeneic melanoma cells can induce objective clinical responses and MART-1 specific CD8+T-cell immunity. J Immunother, 2006. 29(5): p. 545-57.
49. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
50. Guo, J., et al., Intratumoral injection of dendritic cells in combination with local hyperthermia induces systemic antitumor effect in patients with advanced melanoma. Int J Cancer, 2007. 120(11): p. 2418-25.
51. O'Rourke, M. G., et al., Dendritic cell immunotherapy for stage IV melanoma. Melanoma Res, 2007. 17(5): p. 316-22.
52. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
53. Hersey, P., et al., Phase I/II study of treatment with matured dendritic cells with or without low dose IL-2 in patients with disseminated melanoma. Cancer Immunol Immunother, 2008. 57(7): p. 1039-51.
54. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
55. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
56. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.
57. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
58. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
59. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
60. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
61. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
62. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
63. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
64. Ribas, A., et al., Multicenter phase II study of matured dendritic cells pulsed with melanoma cell line lysates in patients with advanced melanoma. J Transl Med, 2010. 8: p. 89.
65. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
66. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
67. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
68. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
69. Steele, J. C., et al., Phase I/II trial of a dendritic cell vaccine transfected with DNA encoding melan A and gp100 for patients with metastatic melanoma. Gene Ther, 2011. 18(6): p. 584-93.
70. Kim, D. S., et al., Immunotherapy of malignant melanoma with tumor lysate-pulsed autologous monocyte-derived dendritic cells. Yonsei Med J, 2011. 52(6): p. 990-8.
71. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
72. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
73. Dannull, J., et al., Melanoma immunotherapy using mature DCs expressing the constitutive proteasome. J Clin Invest, 2013. 123(7): p. 3135-45.
74. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
75. Stift, A., et al., Dendritic cell vaccination in medullary thyroid carcinoma. Clin Cancer Res, 2004. 10(9): p. 2944-53.
76. Kuwabara, K., et al., Results of a phase I clinical study using dendritic cell vaccinations for thyroid cancer. Thyroid, 2007. 17(1): p. 53-8.
77. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
78. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
79. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
80. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
81. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
82. Yamanaka, R., et al., Clinical evaluation of dendritic cell vaccination for patients with recurrent glioma: results of a clinical phase I/II trial. Clin Cancer Res, 2005. 11(11): p. 4160-7.
83. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
84. Walker, D. G., et al., Results of a phase I dendritic cell vaccine trial for malignant astrocytoma: potential interaction with adjuvant chemotherapy. J Clin Neurosci, 2008. 15(2): p. 114-21.
85. Leplina, O. Y., et al., Use of interferon-alpha-induced dendritic cells in the therapy of patients with malignant brain gliomas. Bull Exp Biol Med, 2007. 143(4): p. 528-34.
86. De Vleeschouwer, S., et al., Postoperative adjuvant dendritic cell-based immunotherapy in patients with relapsed glioblastoma multiforme. Clin Cancer Res, 2008. 14(10): p. 3098-104.
87. Ardon, H., et al., Adjuvant dendritic cell-based tumour vaccination for children with malignant brain tumours. Pediatr Blood Cancer, 2010. 54(4): p. 519-25.
88. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
89. Okada, H., et al., Induction of CD8+T-cell responses against novel glioma-associated antigen peptides and clinical activity by vaccinations with [alpha]-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
90. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
91. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
92. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
93. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.
94. Fong, B., et al., Monitoring of regulatory T cell frequencies and expression of CTLA-4 on T cells, before and after DC vaccination, can predict survival in GBM patients. PLoS One, 2012. 7(4): p. e32614.
95. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
96. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
97. Akiyama, Y., et al., alpha-type-1 polarized dendritic cell-based vaccination in recurrent high-grade glioma: a phase I clinical trial. BMC Cancer, 2012. 12: p. 623.
98. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
99. Shah, A. H., et al., Dendritic cell vaccine for recurrent high-grade gliomas in pediatric and adult subjects: clinical trial protocol. Neurosurgery, 2013. 73(5): p. 863-7.
100. Reichardt, V. L., et al., Idiotype vaccination using dendritic cells after autologous peripheral blood stem cell transplantation for multiple myeloma—a feasibility study. Blood, 1999. 93(7): p. 2411-9.
101. Lim, S. H. and R. Bailey-Wood, Idiotypic protein-pulsed dendritic cell vaccination in multiple myeloma. Int J Cancer, 1999. 83(2): p. 215-22.
102. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
103. Reichardt, V. L., et al., Idiotype vaccination of multiple myeloma patients using monocyte-derived dendritic cells. Haematologica, 2003. 88(10): p. 1139-49.
104. Guardino, A. E., et al., Production of myeloid dendritic cells (DC) pulsed with tumor-specific idiotype protein for vaccination of patients with multiple myeloma. Cytotherapy, 2006. 8(3): p. 277-89.
105. Lacy, M. Q., et al., Idiotype-pulsed antigen-presenting cells following autologous transplantation for multiple myeloma may be associated with prolonged survival. Am J Hematol, 2009. 84(12): p. 799-802.
106. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.
107. Rollig, C., et al., Induction of cellular immune responses in patients with stage-I multiple myeloma after vaccination with autologous idiotype-pulsed dendritic cells. J Immunother, 2011. 34(1): p. 100-6.
108. Zahradova, L., et al., Efficacy and safety of Id-protein-loaded dendritic cell vaccine in patients with multiple myeloma—phase II study results. Neoplasma, 2012. 59(4): p. 440-9.
109. Timmerman, J. M., et al., Idiotype-pulsed dendritic cell vaccination for B-cell lymphoma: clinical and immune responses in 35 patients. Blood, 2002. 99(5): p. 1517-26.
110. Maier, T., et al., Vaccination of patients with cutaneous T-cell lymphoma using intranodal injection of autologous tumor-lysate-pulsed dendritic cells. Blood, 2003. 102(7): p. 2338-44.
111. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
112. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
113. Li, L., et al., Immunotherapy for patients with acute myeloid leukemia using autologous dendritic cells generated from leukemic blasts. Int J Oncol, 2006. 28(4): p. 855-61.
114. Roddie, H., et al., Phase I/II study of vaccination with dendritic-like leukaemia cells for the immunotherapy of acute myeloid leukaemia. Br J Haematol, 2006. 133(2): p. 152-7.
115. Litzow, M. R., et al., Testing the safety of clinical-grade mature autologous myeloid DC in a phase I clinical immunotherapy trial of CML. Cytotherapy, 2006. 8(3): p. 290-8.
116. Westermann, J., et al., Vaccination with autologous non-irradiated dendritic cells in patients with bcr/abl+ chronic myeloid leukaemia. Br J Haematol, 2007. 137(4): p. 297-306.
117. Hus, I., et al., Vaccination of B-CLL patients with autologous dendritic cells can change the frequency of leukemia antigen-specific CD8+ T cells as well as CD4+CD25+ FoxP3+ regulatory T cells toward an antileukemia response. Leukemia, 2008. 22(5): p. 1007-17.
118. Palma, M., et al., Development of a dendritic cell-based vaccine for chronic lymphocytic leukemia. Cancer Immunol Immunother, 2008. 57(11): p. 1705-10.
119. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
120. Iwashita, Y., et al., A phase I study of autologous dendritic cell-based immunotherapy for patients with unresectable primary liver cancer. Cancer Immunol Immunother, 2003. 52(3): p. 155-61.
121. Lee, W. C., et al., Vaccination of advanced hepatocellular carcinoma patients with tumor lysate-pulsed dendritic cells: a clinical trial. J Immunother, 2005. 28(5): p. 496-504.
122. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
123. Palmer, D. H., et al., A phase II study of adoptive immunotherapy using dendritic cells pulsed with tumor lysate in patients with hepatocellular carcinoma. Hepatology, 2009. 49(1): p. 124-32.
124. El Ansary, M., et al., Immunotherapy by autologous dendritic cell vaccine in patients with advanced HCC. J Cancer Res Clin Oncol, 2013. 139(1): p. 39-48.
125. Tada, F., et al., Phase I/II study of immunotherapy using tumor antigen-pulsed dendritic cells in patients with hepatocellular carcinoma. Int J Oncol, 2012. 41(5): p. 1601-9.
126. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
127. Hirschowitz, E. A., et al., Autologous dendritic cell vaccines for non-small-cell lung cancer. J Clin Oncol, 2004. 22(14): p. 2808-15.
128. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
129. Yannelli, J. R., et al., The large scale generation of dendritic cells for the immunization of patients with non-small cell lung cancer (NSCLC). Lung Cancer, 2005. 47(3): p. 337-50.
130. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
131. Antonia, S. J., et al., Combination of p53 cancer vaccine with chemotherapy in patients with extensive stage small cell lung cancer. Clin Cancer Res, 2006. 12(3 Pt 1): p. 878-87.
132. Perrot, I., et al., Dendritic cells infiltrating human non-small cell lung cancer are blocked at immature stage. J Immunol, 2007. 178(5): p. 2763-9.
133. Hirschowitz, E. A., et al., Immunization of NSCLC patients with antigen-pulsed immature autologous dendritic cells. Lung Cancer, 2007. 57(3): p. 365-72.
134. Baratelli, F., et al., Pre-clinical characterization of GMP grade CCL21-gene modified dendritic cells for application in a phase I trial in non-small cell lung cancer. J Transl Med, 2008. 6: p. 38.
135. Hegmans, J. P., et al., Consolidative dendritic cell-based immunotherapy elicits cytotoxicity against malignant mesothelioma. Am J Respir Crit Care Med, 2010. 181(12): p. 1383-90.
136. Um, S. J., et al., Phase I study of autologous dendritic cell tumor vaccine in patients with non-small cell lung cancer. Lung Cancer, 2010. 70(2): p. 188-94.
137. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
138. Perroud, M. W., Jr., et al., Mature autologous dendritic cell vaccines in advanced non-small cell lung cancer: a phase I pilot study. J Exp Clin Cancer Res, 2011. 30: p. 65.
139. Skachkova, O. V., et al., Immunological markers of anti-tumor dendritic cells vaccine efficiency in patients with non-small cell lung cancer. Exp Oncol, 2013. 35(2): p. 109-13.
140. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
141. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
142. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
143. Kandalaft, L. E., et al., A Phase I vaccine trial using dendritic cells pulsed with autologous oxidized lysate for recurrent ovarian cancer. J Transl Med, 2013. 11: p. 149.
144. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
145. Rong, Y., et al., A phase I pilot trial of MUC1-peptide-pulsed dendritic cells in the treatment of advanced pancreatic cancer. Clin Exp Med, 2012. 12(3): p. 173-80.
146. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.

1. A method of preventing or treating cancer through administration of one or more mRNA molecules encoding the whole or a portion of the survivin gene, wherein said mRNA are survivin modified mRNA.

2. The method of claim 1, wherein the modified mRNA contains at least one chemically modified nucleoside selected from the group consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propyny1-uridine, 1-propynyl-pseudouridine, 5-taurinom-ethyluridine, 1-taurinomethyl-pseudouridine, 5-taurinom-ethyl-2-thio-uridine, 1-taurinomethyl-4-thio-uridine, 5-methyl-uridine, 1-methylpseudouridine, 4-thio-1-methyl-pseudouridine, 2-thio-1-methyl-pseudouridine, 1-methyl-1-deaza-pseudouridine, 2-thio-1-methyl-1-deaza-pseudouri-dine, dihydrouridine, dihydropseudouridine, 2-thio-dihydrouridine, 2-thio-dihydropseudouridine, 2-methoxyuridine, 2-methoxy-4-thio-uridine, 4-methoxypseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thiozebularine, 2-thio-zebularine, 2-methoxy-cytidine, 2-methoxy-5-methyl-cytidine, 4-methoxy-pseudoisocytidine 4-methoxy-1-methy 1-pseudoisocytidine, 2-aminopurine, 2,6-diaminopurine, 7-deaza-adenine, 7-deaza-8-azaadenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyl-adenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deazaguanosine, 6-thio-7-deaza-8-aza-guanosine, 7-methylguanosine, 6-thio-7-methyl-guanosine, 7-methylinosine, 6-methoxy-guanosine, 1-methylguanosine, N2-methyl-guanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

3. The method of claim 1, wherein said administration is performed by subcutaneous injection of said mRNA molecules.

4. The method of claim 3, wherein said administration of said mRNA molecules is performed into the lymph nodes.

5. The method of claim 4, wherein agents are administered prior to administration of mRNA in order to enhance immunogenicity of molecules transcribed from said mRNA.

6. The method of claim 5, wherein said agent capable of enhancing immunogenicity of said molecules transcribed from said mRNA is G-CSF.

7. The method of claim 5, wherein said agent capable of enhancing immunogenicity of said molecules transcribed from said mRNA is GM-CSF.

8. The method of claim 5, wherein said agent capable of enhancing immunogenicity of said molecules transcribed from said mRNA is M-CSF.

9. The method of claim 5, wherein said agent capable of enhancing immunogenicity of said molecules transcribed from said mRNA is interleukin-3.

10. The method of claim 5, wherein said agent capable of enhancing immunogenicity of said molecules transcribed from said mRNA is M-CSF and interferon gamma.

11. The method of claim 5, wherein said agent capable of enhancing immunogenicity of said molecules transcribed from said mRNA is GM-CSF and interferon gamma.

12. The method of claim 5, wherein said agent capable of enhancing immunogenicity of said molecules transcribed from said mRNA is G-CSF and interferon gamma.

13. The method of claim 5, wherein said agent capable of enhancing immunogenicity of said molecules transcribed from said mRNA is G-CSF and interferon gamma and antibody to interleukin-10.

14. The method of claim 5, wherein said agent capable of enhancing immunogenicity of said molecules transcribed from said mRNA is GM-CSF and interferon gamma and antibody to interleukin-10.

15. The method of claim 5, wherein said agent capable of enhancing immunogenicity of said molecules transcribed from said mRNA is M-CSF and interferon gamma and antibody to interleukin-10.

16. The method of claim 5, wherein said agent capable of enhancing immunogenicity molecules transcribed from said mRNA is M-CSF and antibody to interleukin-10.

17. The method of claim 5, wherein said agent capable of enhancing immunogenicity molecules transcribed from said mRNA is G-CSF and antibody to interleukin-10.

18. The method of claim 5, wherein said agent capable of enhancing immunogenicity molecules transcribed from said mRNA is GM-CSF and antibody to interleukin-10.

19. The method of claim 5, wherein said agent capable of enhancing immunogenicity molecules transcribed from said mRNA is interleukin-4.

20. The method of claim 5, wherein said agent capable of enhancing immunogenicity molecules transcribed from said mRNA is interleukin-2.
